(19) United States
(12) Patent Application Publication
(10) Pub. No.: US 2004/0048871 A1
(43) Pub. Date: Mar. 11, 2004

(54) Use of high dose intravenous methotrexate, with leucovorin rescue, to treat early multiple sclerosis and other diseases of the central nervous system

(76) Inventor: Vernon D. Rowe, Kansas City, MO (US)

Correspondence Address: STINSON MORRISON HECKER LLP, ATTN: PATENT GROUP, 1201 WALNUT STREET, SUITE 2800, KANSAS CITY, MO, 64106-2150, US

(21) Appl. No.: 10/237,320

(22) Filed: Sep. 9, 2002

(51) Int. Cl. A61K 31/525; A61K 38/21

(52) U.S. Cl. 514/251; 514/2

(57) ABSTRACT

The present invention is directed to a method for treating a non-infectious non-neoplastic inflammatory condition of the CNS comprising administering an initial dose of methotrexate to a human host exhibiting a symptom of said condition at a level which is sufficient to cross the blood brain barrier, wherein said human host has not been clinically diagnosed with said condition, or has not been treated with an immunomodulatory agent. The high dose methotrexate treatment is followed by rescue of the periphery with a methotrexate inhibitor that does not cross the blood brain barrier, and administration of a follow-up treatment considered to have therapeutic value for treating said condition.

### CROSS-REFERENCE TO RELATED APPLICATIONS

Not Applicable

### STATEMENT REGARDING FEDERALLY SPONSORED RESEARCH OR DEVELOPMENT

Not Applicable.

### BACKGROUND OF THE INVENTION

1. Field of the Invention

The present invention is directed to the treatment of non-infectious, non-neoplastic inflammatory conditions of the central nervous system using high dose methotrexate treatments.

2. Descripton of Related Art

Multiple sclerosis (MS) is a demyelinating disease of the central nervous system (CNS) with unknown cause and no known cure. Though single episodes of demyelination can occur, once the disease is established in multiple sites in the brain, spinal cord, and optic nerves, MS frequently follows a clinically relapsing-remitting course while lesions in the CNS continue to progress. During this phase, an immune mediated inflammatory response to myelin antigens is thought to play a major role in the pathogenesis of developing lesions. Then, in a clinically progressive phase, at least fifty-five percent of patients worsen, with clinical relapses sometimes punctuating their clinical decline.

The mechanism of tissue damage to the CNS is not known with certainty in the progressive phase of MS. It is thought, however, that axonal damage, perhaps through some type of immune mediation, is important in this phase of the disease, though some axonal damage certainly occurs during the inflammatory phase.

Perivascular infiltration of T lymphocytes and macrophages in brain lesions is one of the characteristics of MS. Activation of myelin-reactive T cells in the periphery is an early event in the MS process. These activated T cells facilitate the production by B cells of antibodies against myelin, and activate macrophages to attack oligodendrocytes in the CNS. The functions of these immune cells are regulated by cytokines in autocrine and paracrine fashions. Proinflammatory cytokines like IFN-γ, and TNF-α could have disease-promoting roles in MS, whereas anti-inflammatory cytokines, like IL-4, IL-10 and TGF-β, likely down-regulate the disease. The balance between pro-inflammatory and anti-inflammatory cytokines may determine the outcome of injury in MS.

Treatment options for patients with MS are limited. Currently, the primary drugs used to treat MS are interferons and glatiramer acetate. These drugs are marketed under the brand names AVONEX by Biogen, Inc. (interferon-beta-1a, recombinant), REBIF by Serono, Inc. (interferon-beta-1a), BETASERON by Berlex Laboratories, Inc. (interferon-beta-1b, recombinant) and COPAXONE by Teva Neuroscience, LLC (copolymer-1, glatiramer acetate), and are often referred to as the “ABC” treatments. Such treatments have been shown to slow, but not arrest, the clinical course of progression in progressive MS. Thus, alternative, or backup, treatment methods are needed.

Certain chemotherapeutic agents, such as methotrexate, mitoxantrone and cyclophosphamide, have been used to treat MS. Although mitoxantrone and cyclophosphamide have shown some efficacy against the progression of MS, they do not cross the blood brain barrier (BBB) into the CNS and mitoxantrone has limited lifetime use due to cardiotoxicity.

Methotrexate is an S-phase chemotherapeutic anti-metabolite, used for the treatment of various neoplasms, particularly CNS lymphoma. Methotrexate is also an antiinflammatory agent and has been used for the treatment of various autoimmune diseases, such as rheumatoid arthritis and psoriasis. Methotrexate is a folate analogue which competitively binds and inhibits dihydrofolate reductase (DHFR), and thus inhibits the synthesis of thymidine and other compounds requiring methylation for their synthesis by inhibiting the single carbon transfer necessary for their synthetic pathways. Methotrexate also promotes the release of adenosine, and this mechanism may be responsible for its anti-inflammatory activity.

Clinical trials using low-dose methotrexate (7.5 mg/week), administered orally, in progressive MS, have been shown to impact the course of progressive MS with minimal toxicity. This treatment option has been widely adopted in the United States for MS patients in the progressive phase who are developing upper extremity dysfunction. Some MS centers empirically treat refractory patients with higher doses (15 mg or 20 mg) orally once a week. However, when administered orally, the serum level of methotrexate is not sufficient to cross the BBB in cytotoxic amounts. Thus, while oral methotrexate treatments show mild improvement in upper extremity strength in MS patients, a need remains for a treatment that arrests or reverses the progression of the disease.

Methotrexate has been given intravenously in high enough doses to cross the BBB and enter the CNS. The peripheral bone marrow, immune system, gastrointestinal endothelium and other vital rapidly dividing tissues can be rescued by an inhibitor of methotrexate, such as leucovorin, which does not penetrate the BBB. The safety of high dose methotrexate therapy with leucovorin rescue has been demonstrated. For example, clinical trials using high dose methotrexate (8 g/m²), administered via a four hour intravenous (IV) infusion, with leucovorin rescue, have shown promising results in treating CNS lymphomas with low toxicity. High dose methotrexate (2.5 g/m²) sporadically administered via IV infusion, with leucovorin rescue, has similarly shown no significant toxicity. Further, it is believed no cumulative deficit from repeated treatments has been reported.

There are no tests that can, by themselves, determine if a person has multiple sclerosis. However, standard criteria have been established for diagnosing MS. These criteria require that there be objective evidence of at least two neurological events consistent with demyclination, separated in time, to diagnose clinically definite MS. Demyelinating events currently used in diagnosing MS include optic neuritis, incomplete transverse myelitis and syndrome of the brain stem or cerebellum. The demyelinating events must be separated in time and there must be no other explanation for the events or the symptoms the person is experiencing. MRI and other tests can assist in confirming the diagnosis. Regardless of what other tests may be conducted, under standard criteria, a clinically definite diagnosis of MS is not made until at least two (2) demyelinating events, separated in time, have been observed.

### BRIEF SUMMARY OF THE INVENTION

The present invention is directed to a method for treating a non-infectious non-neoplastic inflammatory condition of the CNS comprising administering an initial dose of methotrexate to a human host exhibiting a symptom of said condition at a level which is sufficient to cross the blood brain barrier, wherein said human host has not been clinically diagnosed with said condition, or has not been treated with an immunomodulatory agent. The high dose methotrexate treatment is followed by rescue of the periphery with a methotrexate inhibitor that does not cross the blood brain barrier, and administration of a follow-up treatment considered to have therapeutic value for treating said condition.

### DETAILED DESCRIPTION OF PREFERRED EMBODIMENT

The present invention provides a method for treating MS, and other non-infectious, non-neoplastic inflammatory conditions of the CNS, by administering an initial high dose of methotrexate in an amount sufficient to cross the BBB after the first symptoms of the condition are presented and/or prior to treatment with other immunomodulatory agents. The initial high dose methotrexate treatment is preferably followed by additional follow-up treatments considered to have therapeutic value in treating the condition.

The initial high dose methotrexate treatment is preferably administered as soon as possible after the patient exhibits symptoms of the CNS condition, preferably before the condition has been clinically diagnosed, and more preferably after the first occurrence of the symptom. Alternatively, the high dose methotrexate treatment may be administered after the condition has been clinically diagnosed, but before the patient has been treated with other immunomodulatory agents. It is believed that methotrexate can purge the CNS of immune cells responsible for the destructive inflammatory process involved in disease formation, thereby arresting or inhibiting progression of the disease. By administering the high dose methotrexate early in the disease progression, it is believed there is a better chance of completely purging the CNS of the destructive immune cells and preventing their return.

The initial high dose methotrexate is administered by IV infusion at a dosage sufficient to cross the BBB and enter the CNS at a cytotoxic level. Preferably the initial high dose methotrexate treatment is administered as a single bolus dose. However, the initial high dose methotrexate may be administered in multiple doses, provided each dose is sufficient to cross the BBB and enter the CNS at a cytotoxic level, and is followed by leucovorin rescue of the periphery. Thus, it should be understood that as used herein, the term “initial high dose” may refer to a singe bolus dose or multiple doses that make up a cumulative “initial high dose.” Preferably, each administration comprising the initial high dose is between about 0.5 and about 4.0 g/m², more preferably between about 1.0 and 4.0 g/m², and most preferably is about 2.0 g/m². However, lower doses of methotrexate may be used to achieve the effect of an initial high dose treatment if combined with agents and/or treatments which lower the BBB.

After administration of the high dose methotrexate, the peripheral bone marrow, immune system, gastrointestinal endothelium and other vital rapidly dividing tissues can be rescued by an inhibitor of methotrexate, such as leucovorin, which does not penetrate the BBB in a sufficient amount to inhibit the action of the methotrexate within the CNS.

In patients suffering from an acute demyelinating event at the time of treatment, administration of a methotrexate inhibitor is preferably followed by administration of an antiinflammatory agent, preferably an anti-inflammatory steroid, more preferably a corticosteroid and most preferably methylprednisolone or prednisone. The anti-inflammatory agent is preferably administered via IV for several days, followed by several additional days of oral administration with the same or a different anti-inflammatory agent. In a preferred embodiment, methylprednisolone is administered via IV and prednisone is administered orally, as can readily be determined by one skilled in the art.

Follow-up treatments considered to have therapeutic value for treating the condition are preferably used to assist in preventing any destructive immune cells from the peripheral immune system from crossing the BBB and repopulating the CNS. The follow-up treatments are preferably given over a period of time. In a preferred embodiment the follow-up treatments are given for at least one year, more preferably at least two years and most preferably for at least three years.

The follow-up treatment is preferably started after the leucovorin rescue, but may begin during the treatment with the anti-inflammatory agent, if employed. If anti-inflammatory agents are employed, the follow-up treatment preferably is started after completion of the Iv anti-inflammatory agent treatments, but while the patient is still taking the oral antiinflammatory agent. The follow-up treatment is preferably administered at dosages and intervals suitable for treatment of the condition.

In a preferred embodiment, the method of the present invention is used to treat MS. In one such embodiment, the high dose methotrexate treatment is administered after the patient is diagnosed with a first demyelinating event and before the patient is diagnosed with a second demyelinating event, i.e. before development of clinically definite MS. It is believed that treating the patient after the first demyelinating event may delay or prevent the onset of clinically definite MS. In the preferred embodiment, the initial high dose methotrexate treatment is preferably administered within three (3) months, more preferably twenty-one (21) days, and most preferably within twelve (12) days of the onset of the first demyelinating event.

In a second embodiment wherein the method of the present invention is used to treat MS, the high dose methotrexate treatment is administered after the second demyelinating event, i.e. after a clinical diagnosis, but prior to beginning treatment with an immunomodulatory agent. In a third such embodiment, a single high dose of methotrexate may be administered after a clinical diagnosis and after treatment with an immunomodulatory agent, preferably before the patient has failed to respond to the immunomodulatory agent.

As used herein, demyelinating event is used to refer to any event consistent with possible MS. Demyelinating events may include events consistent with demyelination with the optic nerve (optic neuritis), spinal cord (incomplete transverse myelitis), or brain stem or cerebellum (brain stem or cerebellar syndrome) or any event determined in the future to be consistent with possible MS. Most preferably characterization of the event as a demyelinating event will be supported by the appearance two or more clinically silent lesions on the brain that are at least 3 mm in diameter on MRI scans and are characteristic of MS in that at least one lesion is periventricular or ovoid. Supportive evidence may also be a cerebral spinal fluid exam characteristic of MS.

The high dose methotrexate treatment is followed by additional follow-up treatments considered to have therapeutic value for treating MS. Preferably, the high dose methotrexate treatment is followed by treatment with a therapeutic agent considered to have therapeutic value in treating MS, more preferably an immunomodulatory agent that is considered to have therapeutic value in treating MS, and even more preferably the immunomodulatory agent is selected from the group consisting of interferon, glatiramer acetate and methotrexate. The immunomodulatory agents may be administered at dosages and intervals suitable for the treatment of relapsing MS, as can be determined by one in the art.

The follow-up treatment is preferably started after leucovorin rescue and any IV methylprednisone treatment, more preferably within fifteen (15) days of the high dose methotrexate treatment. Preferably the follow-up treatments may be administered outside of a hospital setting by the patient or caregiver.

The following example more fully illustrates one embodiment of the present invention:

### EXAMPLE 1

Approximately twenty (20) subjects will be recruited for a study. To be eligible for the study, patients will preferably have had a first isolated, well-defined neurologic event consistent with demyelination and involving the optic nerve (unilateral optic neuritis), spinal cord (incomplete transverse myelitis), or brain stem or cerebellum (brain stem or cerebellar syndrome). They will also preferably have two (2) or more clinically silent lesions of the brain that are at least three (3) mm in diameter on MRI scans and characteristic of MS in that at least one (1) lesion must be periventricular or ovoid. The onset of the symptoms will preferably have occurred no more than twelve (12) days before beginning treatment. Preferably subjects will be between the ages of eighteen (18) and fifty (50), inclusive.

The study may be performed according to the following protocol:

Prior to administration of the initial methotrexate treatment, a urine pH of greater than 7.0 and specific gravity of less than 1.1010 is obtained and the patient is hydrated. An anti-emetic such as ZOFRAN® (ondansetron hydrochloride) is administered by IV at a dosage of 0.15 mg/kg thirty (30) minutes before the methotrexate infusion and may be repeated at four (4) and eight (8) hours post infusion. An anti-emetic may be administered in oral form following discharge to the home.

An initial methotrexate treatment is given within twelve (12) days of the first demyelinating event. Methotrexate is administered by IV at a dosage of 2 g/m² in 200 cc NS over two (2) hours.

The initial methotrexate treatment is followed by IV leucovorin rescue, with 50 mg leucovorin administered exactly eight (8) hours after the start of the methotrexate infusion. Oral leucovorin (50 mg) is administered every six (6) hours for a total of twelve (12) doses, with the first oral dose starting two (2) hours after the leucovorin infusion. If necessary, oral leucovorin administration is continued until the serum methotrexate concentration reaches 0.05 uMOL/L.

Methylprednisolone treatment is commenced the day following methotrexate treatment. Patients are treated with intravenous methylprednisolone 1 gram per day for three (3) days, followed by 1 mg/kg oral prednisone per day for eleven (11) days, and at the end of the eleven (11) days, a taper consisting of 20 mg the first day and 10 mg the second day is administered.

Treatment with interferon beta 1a, glitiramir acetate, or other immunomodulatory agent begins after the completion of the IV methylprednisolone, while the patient is still on oral prednisone. The interferon, glitiramir acetate or other immunomodulatory agents may be administered as appropriate for treating relapsing MS, according to the manufacturer's instruction.

Progression of the disease and onset of clinically definite MS may be monitored using the Expanded Disability Status Scale and the Multiple Sclerosis Functional Composite Score and its subset measures consisting of the Timed 25-foot Walk, Nine-Hole Peg Test an Paced Auditory Serial Addition Test. MRI lesions may also be monitored, as well as the expression of activation markers and intracellular cytokines in peripheral blood T lymphocytes.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, is to be understood that all matters herein set forth are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

What is claimed and desired to be secured by Letters Patent is as follows:

1. A method for treating a non-infectious non-neoplastic inflammatory condition of the CNS comprising:
administering to a human host exhibiting a symptom of said condition an initial dose of methotrexate sufficient to cross the blood brain barrier at a time when said human host has not yet been clinically diagnosed with said condition; and
administering to said host a follow-up treatment considered to have therapeutic value for treating said condition.

2. The method as claimed in claim 1, wherein said follow-up treatment is administered for at least one year.

3. The method as claimed in claim 1, wherein said administering of methotrexate is conducted after the first occurrence of said symptom.

4. The method as claimed in claim 3, wherein said administering of methotrexate is conducted within twelve days after the first occurrence of said symptom.

5. The method as claimed in 1, wherein said administering follow-up treatment step comprises administering a therapeutic agent considered to have therapeutic value for treating said condition.

6. The method as claimed in claim 5, wherein said therapeutic agent is an immunomodulatory agent.

7. The method as claimed in claim 6, wherein said administering of methotrexate is conducted within twelve days after the first occurrence of said symptom.

8. A method for treating multiple sclerosis comprising:
administering to a human host diagnosed with a first demyelinating event an initial dose of methotrexate sufficient to cross the blood brain barrier at a time when said host has not yet been diagnosed with a second demyelinating event; and
administering to said human host a follow-up treatment considered to have therapeutic value for treating multiple sclerosis.

9. The method as claimed in claim 8, wherein said administering of methotrexate is conducted within twelve days after the onset of said first demyelinating event.

10. The method as claimed in 8, wherein said administering follow-up treatment step comprises administering a therapeutic agent considered to have therapeutic value for treating multiple sclerosis.

11. The method as claimed in claim 10, wherein said therapeutic agent is an immunomodulatory agent.

12. The method as claimed in claim 11, wherein said administering of methotrexate is conducted within twelve days after the onset of said first demyelinating event.

13. The method as claimed in claim 11, wherein said therapeutic agent is selected from the group consisting of interferon, glatiramer acetate and methotrexate.

14. The method as claimed in claim 8, further comprising, after said administering of methotrexate, the step of administering an anti-inflammatory agent.

15. The method as claimed in claim 14, further comprising, after said administering of methotrexate, the step of administering a methotrexate inhibitor that does not penetrate the blood brain barrier.

16. The method as claimed in claim 8, wherein said initial dose of methotrexate ranges from about 1 g/m² to about 4 g/m².

17. The method as claimed in claim 16, wherein initial dose of methotrexate is about 2 g/m².

18. The method as claimed in claim 8, wherein said follow-up treatment is administered for at least one year.

19. A method for treating multiple sclerosis comprising:
administering to a human host diagnosed with clinically definite multiple sclerosis an initial dose of methotrexate sufficient to cross the blood brain barrier at a time when said host has not yet been treated with an immunomodulatory agent; and
administering to said human host a follow-up treatment considered to have therapeutic value for treating multiple sclerosis.

20. The method as claimed in claim 19, wherein said administering follow-up treatment step comprises administering an immunomodulatory agent.

21. The method as claimed in claim 19, wherein said initial dose of methotrexate ranges from about 1 g/m² to about 4 g/m².
